(19) United States
(12) Patent Application Publication
(10) Pub. No.: US 2021/0244650 A1
(43) Pub. Date: Aug. 12, 2021

(54) MASK SUBSTRATE AND KIT INCLUDING THE SAME

(71) Applicant: MAXIGEN BIOTECH INC., Taoyuan City (TW)

(72) Inventors: Sung-Ching Chen, Taoyuan City (TW); Yu-Cheng Chang, Taoyuan City (TW)

(21) Appl. No.: 16/906,434

(22) Filed: Jun. 19, 2020

(30) Foreign Application Priority Data
Feb. 11, 2020 (TW) 109104256

(51) Int. Cl. A61K 8/9789; A61K 8/02; A61Q 19/08

(52) U.S. Cl. CPC A61K 8/9789; A61K 8/0279; A61Q 19/08; B65D 85/70; A61K 2800/87; A61K 2800/412; A61K 2800/84

(57) ABSTRACT

A mask substrate and a kit including the same are provided. The mask substrate includes a substrate layer; the substrate layer is prepared by freeze-drying a plant slice having a predetermined thickness under a predetermined condition. After the mask substrate is rehydrated with an infiltrating liquid, a surface of the mask substrate is able to be applied to the skin of an individual. In one of the aspects, the mask substrate may effectively absorb water or essence liquid through a natural fiber pore structure, which may release active substances contained in the substrate to improve the skin moisturizing effect.

### CROSS-REFERENCE TO RELATED APPLICATION

This application claims priority from Taiwan Patent Application No. 109104256, filed on Feb. 11, 2020, in the Taiwan Intellectual Property Office, the content of which is hereby incorporated by reference in its entirety for all purposes.

### BACKGROUND

### 1. Technical Field

The present disclosure relates to a mask substrate, particularly to a mask substrate and kit including the same made from an entire plant slice.

### 2. Description of the Related Art

To improve skin moisturization and whitening effect, people nowadays often apply sheet materials containing essence liquid, skin care liquid, etc., to a skin surface. This is performed to easily maintain the skin in a high-moisturizing state so as to improve the absorbability of the skin to skin care ingredients. Therefore, various types of mask products have been developed in recent years.

Currently, woven or non-woven fabric substrates made via weaving, knitting, or non-woven fabric processing artificial fibers, natural fibers (e.g., cotton), bio-fibers, etc., with great water absorption are often used as mask substrates. However, the substrates made from the aforementioned materials may only be used as carrier layers for essence liquids and cannot be equipped with other functions. Furthermore, since a mask is a disposable product which is discarded after use, problems such as waste and pollution may easily occur.

As mentioned above, in terms of the mask substrate and the manufacturing process thereof, some problems and deficiencies are yet to be solved in the prior art. The present application aims to provide a technical solution that may effectively solve the problems and deficiencies as mentioned above in the prior art.

### SUMMARY

According to one embodiment, the present disclosure provides a mask substrate, including a substrate layer; the substrate layer is prepared by freeze-drying a plant slice having a predetermined thickness under a predetermined condition. After the mask substrate is rehydrated with an infiltrating liquid, a surface of the mask substrate is able to be applied to the skin of an individual.

Preferably, the substrate layer may be a thickness of 0.1 to 3 mm.

Preferably, the predetermined condition for freeze-drying may be a cavity temperature of 25° C. to −60° C., and a vacuum degree of 0 to 200 mT.

Preferably, the plant slice may be selected from a group consisting of white radish, taro, pumpkin, wax gourd, turnip, gherkin, cucumber, loofah, watermelon, pineapple, and carrot.

Preferably, the substrate layer has pores in a size of 5 to 100 μm.

Preferably, the mask substrate further includes a packaging bag which may seal the substrate layer therein.

Preferably, the substrate layer may be made without weaving, knitting, or non-woven fabric processing.

According to one embodiment, the present disclosure provides a mask kit, including a mask substrate and an infiltrating liquid, wherein the infiltrating liquid is deionized water or essence liquid; wherein after the mask substrate is rehydrated with an infiltrating liquid, a surface of the mask substrate is able to be applied to the skin of an individual.

In summary, the mask substrate and the kit including the same in the present disclosure have the following advantages:

(1) In the present disclosure, the mask substrate prepared by using a fresh plant slice via a freeze-drying process has a natural plant fiber pore structure. Therefore, the natural plant fiber pore structure may effectively absorb water or essence liquid to acquire a fine skin moisturizing effect when applied.

(2) The plant slice of the present disclosure is selected from an entire slice of different plants (e.g., gherkin, cucumber, loofah, etc.) which may maintain active substances of the selected plant. Therefore, when the mask substrate is infiltrated with water or essence liquid, active substances may be released. Then, when the mask substrate is applied, the active substances may be effectively absorbed by the skin.

(3) The mask substrate of the present disclosure is made from a natural plant slice, so additional materials that may cause skin irritation, such as artificial fibers, are not required. Hence, allergy may be avoided, and this natural product may be completely biodegradable.

(4) The mask kit of the present disclosure may be used with an infiltrating liquid containing ingredients for moisturizing, anti-aging, or whitening to fit consumer needs, which may further optimize the functions of the mask substrate.

### BRIEF DESCRIPTION OF THE DRAWINGS

The exemplary embodiment of the present disclosure is described in detail with reference to the following drawings:

FIG. 1 is a flowchart of the manufacturing method of the mask substrate of the present disclosure.

FIG. 2 shows images of the mask substrate prepared according to the manufacturing method of the mask substrate of the present disclosure.

FIG. 3 shows images of the mask substrate prepared according to the method Comparative Example 1.

FIG. 4 shows images of the mask substrate prepared according to the method of Comparative Example 2.

FIG. 5 is an image of the mask substrate prepared according to the method of Comparative Example 3.

FIG. 6 is an image of the mask substrate prepared according to the method of Comparative Example 4.

FIG. 7 is an image of the mask kit of the present disclosure.

FIG. 8 is an electron microstructure observation image using a cucumber slice as the mask substrate of the present disclosure.

FIG. 9 is an electron microstructure observation image using a gherkin slice as the mask substrate of the present disclosure.

FIG. 10 shows state photographs using a loofah slice as the mask substrate of the present disclosure.

FIG. 11 shows a state photograph using a gherkin slice as the mask substrate of the present disclosure.

FIG. 12 shows a state photograph using a cucumber slice as the mask substrate of the present disclosure.

### DESCRIPTION OF THE PREFERRED EMBODIMENTS

The present disclosure is to be further described in detail by the following preferred embodiments together with the drawings. It should be noted that the experimental data disclosed in the following embodiments are used to explain the technical features of the present disclosure, and are not intended to limit the aspects to be implemented.

### Definitions

In the present application, the term, “approximately”, is intended to indicate a value including, for example, tolerance in the proportion of materials, tolerance in drug concentration values, or variations existing between experimental subjects. The term typically refers to variability of values equal to approximately or less than 1%, 2%, 3%, 4%, 5%, 6%, 7%, 8%, 9%, 10%, 11%, 12%, 13%, 14%, 15%, 16%, 17%, 18%, 19%, or 20% depending on the situation.

As used herein, the term, “a plant slice”, refers to a thin slice made from a slicing process by using roots, stems, leaves, pulps from plants via a slicing machine. Wherein, the plants may be, for instance, white radish, taro, pumpkin, wax gourd, turnip, gherkin, cucumber, loofah, watermelon, pineapple, and carrot, preferably gherkin, cucumber, and loofah. However, the present disclosure is not limited thereto.

As used herein, the term, “non-woven fabric processing”, refers to a net or fabric having the structure of randomly inter-laid fibers or threads, which is not a method identified under the case of knitting and weaving fabric, however.

As used herein, the terms, “knitting” and “weaving”, refer to the use of a machine, such as a weaving machine or a weaving machine, to enable fibers to be woven and intertwined to form a knitting structure or a weaving structure.

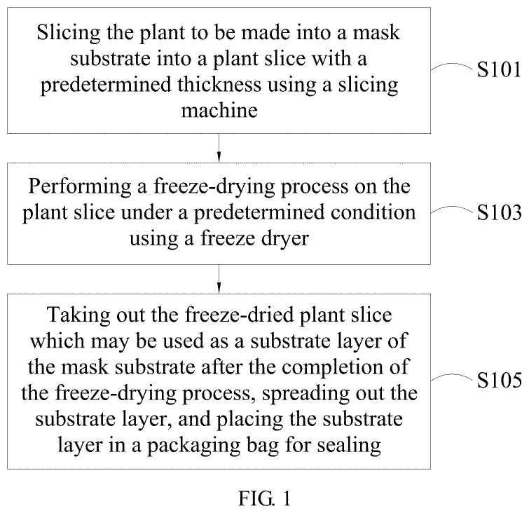

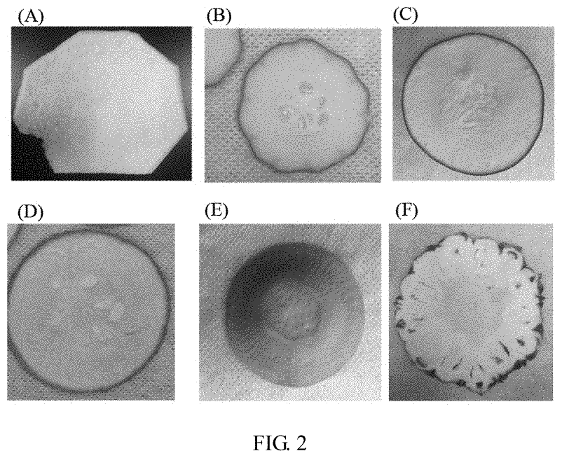

Please refer to FIG. 1 and FIG. 2 which respectively are a flowchart of the manufacturing method of the mask substrate and an image of the mask substrate made from the manufacturing method of the mask substrate of the present disclosure. The manufacturing method of the mask substrate according to the present disclosure includes the following steps: the plant-slicing step (S101): slicing the plant to be made into a mask substrate into a plant slice with a predetermined thickness using a slicing machine, wherein the predetermined thickness may be approximately 0.1 to 3 mm, preferably 0.3 to 2.75 mm, and more preferably 0.5 to 2.5 mm, 0.7 to 2.25 mm, 1 to 2 mm, 1.2 to 1.75 mm, or 1.4 to 1.5 mm; the freeze-drying step (S103): spreading out the plant slice on a stainless steel plate and performing a freeze-drying process under a predetermined condition using a freeze dryer; and the sealing step (S105): taking out the freeze-dried plant slice which may be used as a substrate layer of the mask substrate after the completion of the freeze-drying process, spreading out the substrate layer and placing the substrate layer in a packaging bag for sealing after separated with a separation layer such as a dust-free fabric, and placing it in a cabinet drier for storage to prevent moisture absorption.

In the freeze-drying step (S103), the predetermined conditions for performing the freeze-drying process are: The cavity temperature is approximately 25° C. to −60° C., preferably 20° C. to −45° C., and more preferably 15° C. to −40° C., 10° C. to −35° C., 5° C. to −30° C., 0° C. to −25° C., and −3° C. to −20° C. The vacuum degree is approximately 0 to 200 mT, preferably 25 to 175 mT, and more preferably 50 to 150 mT. The processing time is approximately 18 to 40 hours, preferably 20 to 35 hours, and more preferably 23 to 30 hours.

Specifically, the freeze-drying step (S103) of the present disclosure may be divided into three phases, including the first phase of the freeze-drying process: decreasing the temperature of the plant slice from 25° C. to −25° C., remaining for 90 minutes, then decreasing the temperature of the plant slice from −25° C. to −40° C., and remaining for 30 minutes; the second phase of the freeze-drying process: increasing the temperature of the plant slice from −40° C. to −3° C., wherein the vacuum degree remains at 50 to 150 mT, and the operating time is 20 to 30 hours; and the third phase of the drying procedure: having the plant slice remain for 30 minutes under the condition of the temperature at 25° C. and the vacuum degree at 100 mT after the temperature of the plant slice is increased from −3° C. to 25° C., and then completing the freeze-drying step (S103) after remaining from 90 to 150 minutes at 25° C.

It is worth mentioning that, in the first phase as mentioned above, since the use of the two-stage cooling method may ensure that ice crystals may not grow too fast, which may prevent plant fiber tissues from being damaged, the plant slice after the completion of freeze-drying still has fine water absorption. Furthermore, in the second phase and third phase, the plant slice is able to be dried to a finest state by a precise control of the vacuum degree, temperature, and time. That is, the plant slice prepared by the freeze-drying process of the present disclosure may maintain a structural integrity without having the problems, such as warping, expansion or fracture, etc.

The substrate layer acquired in the manufacturing method for the mask substrate according to the present disclosure is a thin slice directly obtained from the plant slice. Therefore, the substrate layer is made without weaving, knitting, or non-woven fabric processing. As a result, the substrate layer is not mixed with woven fabric, knitted fabric, or non-woven fabric, such as artificial fibers, silk fibers, or biological fibers. Instead, the substrate layer is formed by the natural structure of the plant slice. Wherein, the substrate layer has pores in various sizes formed by the natural structure; the size of the pores may be approximately 5 to 100 μm, preferably 10 to 80 μm, and more preferably 20 to 50 μm.

Please refer to FIG. 2 and Table 1 below, which respectively are the images and the evaluation of the mask substrate prepared according to the manufacturing method of the mask substrate of the present disclosure. In FIG. 2, the images of various plants as the mask substrate are displayed, wherein (A) a wax gourd plant slice is used; (B) a gherkin plant slice is used; (C) a cucumber plant slice is used; (D) a loofah plant slice is used; (E) a carrot plant slice is used; (E) a pineapple plant slice is used; the mask substrates from various plants have flat and complete surfaces. Then, testing personnel performs visual observation and infiltrates the mask substrate with water in order to conduct suitability evaluation of the mask substrate. The result is shown in the table below.

TABLE 1

| | | Suitability |
|---|---|---|
| | | evaluation of |
| Plant | Test result | the substrate |
| White | The freeze-dried slice is fairly flat and has a sponge-like pore | ◯ |
| radish | structure. Although the color is slightly lighter, it still maintains a | |
| | natural color. The slice still maintains a sponge texture after water | |
| | absorption. | |
| Pumpkin | The slice remains the color of the pumpkin after freeze-drying and | ◯ |
| | presents a fairly dense texture. The slice shows a soft form after | |
| | water absorption, has a sponge structure that is tough and not easy | |
| | to tear, and has a fitting feature. | |
| Taro | The slice remains the color of the taro after freeze-drying and | ◯ |
| | presents a fairly dense texture. The slice shows a soft form after | |
| | water absorption, has a sponge structure that is tough and not easy | |
| | to tear, and has a fitting feature. | |
| Wax | The slice presents a loose sponge structure after freeze-drying, | ◯ |
| gourd | easily absorbs water, and shows a soft form after soaked in water. | |
| Turnip | The slice is fluffy after freeze-drying, and is easier to tear with | Δ |
| | hand, but presents better toughness and not easy to tear after soaked | |
| | in water. | |
| Gherkin | The freeze-dried slice is fairly flat and has a sponge-like pore | ⊚ |
| | structure after freeze-drying. Although the color fades slightly, it | |
| | still maintains a natural color. The slice maintains the sponge | |
| | texture after water absorption, and a light gherkin fragrance is | |
| | released. | |
| Loofah | The freeze-dried slice is fairly flat and has a sponge-like pore | ⊚ |
| | structure after freeze-drying. The pores are larger than those of the | |
| | freeze-dried slice of a gherkin. Although the color fades slightly, | |
| | it maintains a natural color. The slice maintains the sponge texture | |
| | after water absorption, and a slight loofah fragrance is released. | |
| | Both a dry slice and a hygroscopic slice have toughness, are very | |
| | soft, and have a fitting feature. | |

⊚: Very suitable to be used as a mask substrate
◯: Suitable to be used as a mask substrate
Δ: Suitable to be used as a mask substrate, yet with relatively little water absorption

From Table 1, it may be known that the mask substrate prepared by the manufacturing method of the mask substrate of the present disclosure may still fully maintain the integrity of the substrate without easily being fractured even under the circumstance of the substrate being very thin. In the meantime, after fully infiltrated in the essence liquid, fine water retention may be well maintained.

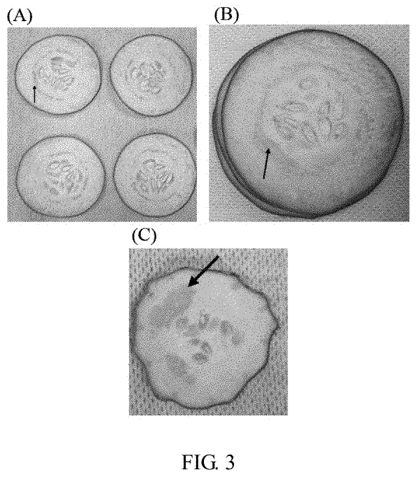

Then, please refer to FIG. 3 to FIG. 6, which respectively are images of the mask substrate prepared according to the methods from Comparative Example 1 to Comparative Example 4. Firstly, the difference of the processing conditions between Comparative Example 1 and the present disclosure being: In the freeze-drying process of the first phase, the temperature of the plant slice is decreased from 25° C. to −25° C. with remaining for 168 minutes, and then the temperature of the plant slice is decreased from −25° C. to −40° C. with remaining for 30 minutes. The rest of conditions are the same as those mentioned above. With reference to the result of FIG. 3, it may be known that (A) and (B) of FIG. 3 show the cucumber mask substrate prepared by the method of Comparative Example 1, and (C) of FIG. 3 shows the gherkin mask substrate prepared by the method of Comparative Example 1. Whether a cucumber or a gherkin is used as the mask substrate, it is obvious that the structure and form of plant fiber tissues are damaged (pointed by the arrows in FIG. 3). This shows that excessive freezing time in the first phase may cause poor tissue structure of plant fibers, and the plant slice may not be able to recover as a fine mask substrate after the mask is rehydrated.

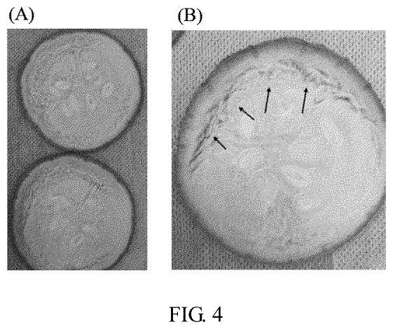

The difference of the processing conditions between Comparative Example 2 and the present disclosure being: In the freeze-drying process of the first phase, the temperature of the plant slice is decreased from 25° C. to −25° C. with remaining for 96 minutes, and then the temperature of the plant slice is decreased from −25° C. to −40° C. with remaining for 30 minutes. The rest of conditions are the same as those mentioned above. With reference to the result of FIG. 4, regarding the loofah mask substrate prepared by the method of Comparative Example 2, it is also obvious that the structure and form of plant fiber tissues are damaged (pointed by the arrows in FIG. 4). Similar to the result of FIG. 3, this shows that excessive freezing time in the first phase may make the plant slice not be able to be used as a fine mask substrate.

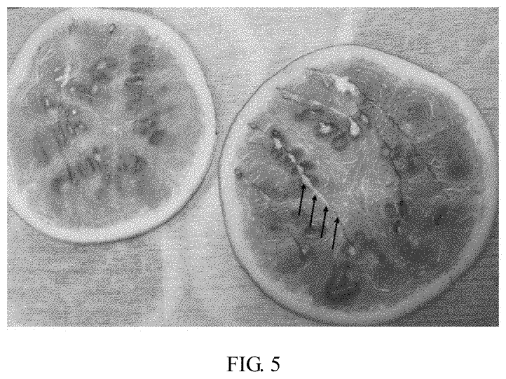

The difference of the processing conditions between Comparative Example 3 and the present disclosure being: In the freeze-drying process of the third phase, the temperature of the plant slice is increased from −3° C. to 25° C., the pressure is restored back to normal pressure, and the temperature of the plant slice remains at 25° C. for 20 minutes. The rest of conditions are the same as those mentioned above. With reference to the result of FIG. 5, regarding the water melon mask substrate prepared by the method of Comparative Example 3, obvious tissue fractures appear (pointed by the arrows in FIG. 5). This may account for the fact that the speeds of returning to normal temperature and returning from vacuum degree back to normal pressure are too fast after freeze-drying the plant slice. This leads to the plant slice failing to maintain structural integrity, further causing fractures.

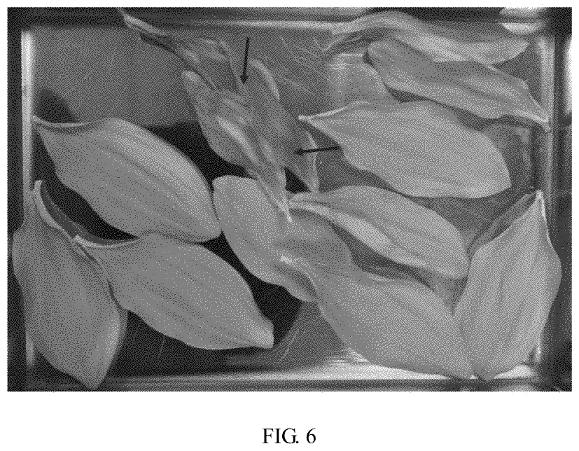

The difference of the processing conditions between Comparative Example 4 and the present disclosure being: In the freeze-drying process of the second phase, the temperature of the plant slice is increased from −40° C. to −3° C., the vacuum degree remains at 150 mT, and the operating time is 5 hours. In the freeze-drying process of the third phase, the temperature of the plant slice is increased from −3° C. to 25° C., the temperature of the plant slice remains for 30 minutes at the temperature of 25° C. and a vacuum degree of 150 mT, and then the temperature of the plant slice remains for 90 to 150 minutes at the temperature of 25° C. The rest of conditions are the same as those mentioned above. With reference to the result of FIG. 6, regarding the sunflower petal mask substrate prepared by the method of Comparative Example 4, a drying problem may be identified (pointed by the arrows in FIG. 6). This may account for the fact that insufficient drying time in the second phase makes the ice inside the plant has not completely dried. This leads to the ice in the frozen part of the plant being warmed to water after the temperature and pressure of the plant slice are returned back to normal temperature and normal pressure. As a result, the plant does not have a complete pore structure after drying.

That is, from Comparative Examples 1 to 4 as mentioned above, it may be known that failing to precisely control vacuum degree, temperature, and time may prevent the plant slice from drying to the finest state, resulting in the plant slice not able to maintain structural integrity. This may generate problems, such as warping, expansion or fracture, etc., and thus the plant slice may not be precisely rehydrated as a complete, flat and flexible substrate mask.

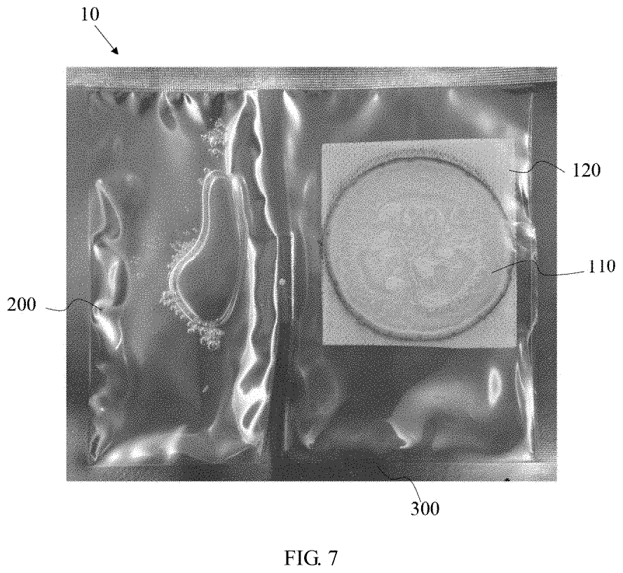

Please refer to FIG. 7 which is an image of the mask kit of the present disclosure. According to the present disclosure, the mask kit includes a mask substrate 110, a separation layer 120, an infiltrating liquid 200, and a packaging bag 300. In one embodiment of the present disclosure, the mask substrate 110 may be used as a plant slice of loofah. However, the present disclosure is not limited thereto. The separation layer 120 may be, for instance, a dust-free cloth woven from polyester fibers. However, the present disclosure is not limited thereto. That is, the separation layer may be any material as long as the mask substrate 110 may be separated without causing adhesion. In one embodiment of the present disclosure, the infiltrating liquid 200 may be deionized water or essence liquid; the infiltrating liquid 200 may contain ingredients for moisturizing, anti-aging, or whitening to fit consumer needs, thus further optimizing the effect of the mask substrate 110.

According to one embodiment of the present disclosure, the packaging bag 300 may be divided into two parts for package. For one part of the package, a plurality of mask substrates 110 are stacked by the separation layer 120, and for the other part of the package, the infiltrating liquid 200 is packaged in a predetermined amount. Hence, the mask substrate 110 is taken out and fully infiltrated with infiltrating liquid 200 before use, and then applied to the skin of an individual. However, the present disclosure is not limited thereto. In another embodiment, the packaging bag 300 may also package the mask substrate 110 and the infiltrating liquid 200 altogether, thus only requires to torn open the packaging bag 300 for immediate use.

### Embodiments

The following tests are conducted by the inventor according to various mask substrate materials prepared by the aforementioned manufacturing method.

Total Organic Carbon (TOC) Test

Objective: Total organic substances released by the mask substrate after soaked in deionized water are tested to determine whether the mask substrate may release the nutrients it contains.

After the gherkin mask substrate (gherkin group (FDPC)) and the cucumber mask substrate (cucumber group (FDP)) with thicknesses of 1 to 2 mm are respectively soaked in deionized water of 80 ml for 20 minutes, the soaked solution and deionized water (DI Water) are detected by a total organic carbon analyzer to conduct a peroxy pyrosulfate heating oxidation/infrared method according to the norms of Taiwan Inspection Technology Co., Ltd. (SGS) (referring to the method of NIEA W543.50C). The result is shown in Table 2 below.

TABLE 2

| Sample name | Total organic carbon (ppm) |
|---|---|
| Control group (DI Water) | 0.11 |
| Gherkin group (FDPC) | 814 |
| Cucumber group (FDP) | 1200 |

From Table 2, it may be known that the TOC test has confirmed that organic substances may be released after the mask substrate is soaked in deionized water in either the gherkin group or the cucumber group according to the mask substrate prepared by the aforementioned manufacturing method.

Water Absorption and pH Test

The non-woven fabric mask, the gherkin mask substrate, the cucumber mask substrate are weighed to gain dry weight. Next, the remaining water is observed after each of the mask substrates is soaked in a known amount of deionized water for 1 minute. The remaining water is subtracted from the known amount of deionized water after water absorption to gain the total amount of water absorption. Further, the gained amount is divided by the dry weight to obtain the amount of water that can be absorbed per gram of the mask substrate. Also, the pH of the moisturized mask substrate is tested. The result is shown in Table 2 below.

TABLE 3

| | Amount of water absorption | |
|---|---|---|
| Sample name | (g/g substrate) | pH value |
| Non-woven fabric mask | 10.6 ± 0.5 | — |
| Gherkin group (FDPC) | 9.5 ± 1.5 | 6-7 |
| Cucumber group (FDP) | 7.7 ± 0.4 | 6-7 |

From Table 3, it may be known that both the gherkin group and the cucumber group have excellent water absorption; especially, the water absorption of the gherkin group has a water absorption effect very similar to that of the non-woven fabric mask commonly used in the market according to the mask substrate prepared by the aforementioned manufacturing method. In addition, both the pH values of the gherkin group and the cucumber group remain neural. Hence, it is unlikely to cause irritation to the skin.

Result of Observation by an Electron Microscope (SEM)

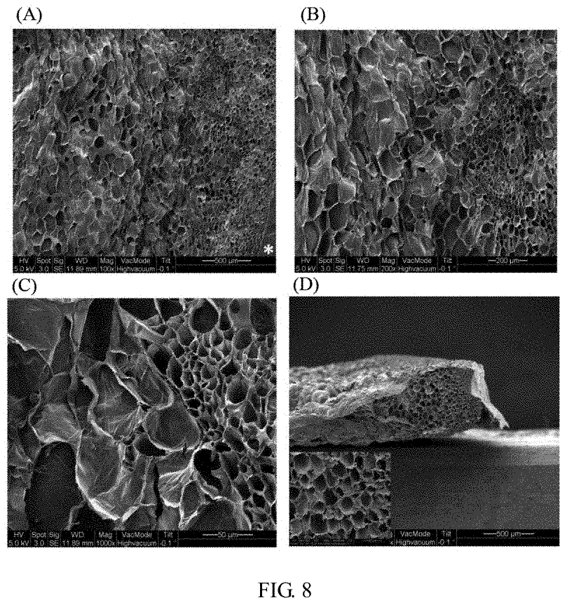

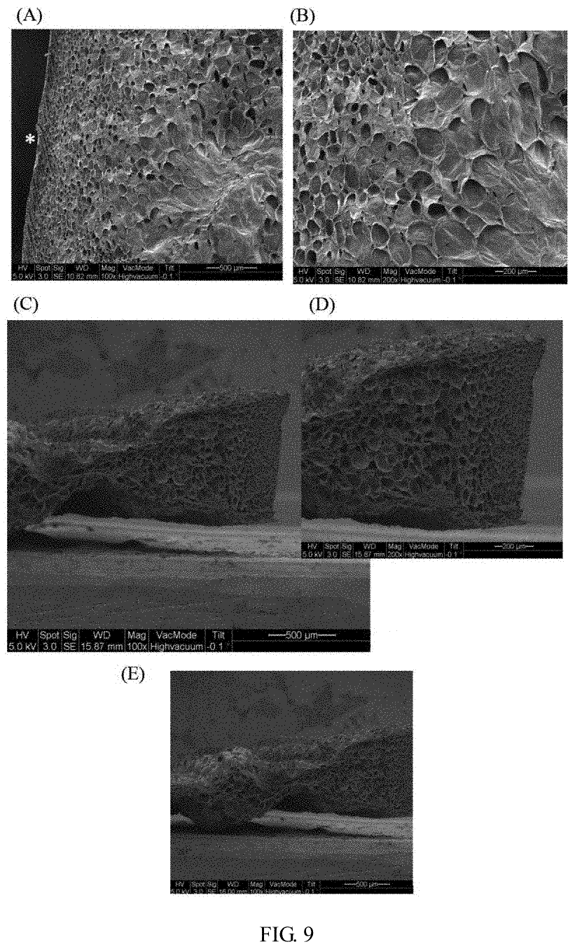

Please refer to FIG. 8 and FIG. 9, which respectively are electron microstructure observation images using a cucumber slice and a gherkin slice as the mask substrates of the present disclosure. In FIG. 8, (A) is a surface image of a cucumber with 500 μm as a proportional scale, wherein * refers to the position of the cucumber's surface; (B) and (C) respectively are surface images of the cucumber with 200 μm and 50 μm as proportional scales; and (D) is a cross-sectional image of a cucumber with 500 μm as a proportional scale. In FIG. 9, (A) is a surface image of a gherkin with 500 μm as a proportional scale, wherein * refers to the position of the gherkin's surface; (B) is a surface image of a gherkin with 200 μm as a proportional scale; (C) and (D) respectively are cross-sectional images of a gherkin with 500 μm and 200 μm as proportional scales; and (E) is a cross-sectional image of a gherkin containing seeds with 500 μm as a proportional scale.

Based on the electron microscope structure images of FIG. 8 and FIG. 9, both the surfaces of the gherkin slice and the cucumber slice have complete pores in different sizes without damages caused by excessive growth of ice crystals. Moreover, the closer to the epidermis, the smaller the pores, and the closer to the pulp, the bigger the pores; the size of the pores is approximately 10 to 50 μm.

Efficacy of Using a Loofah as a Mask Substrate

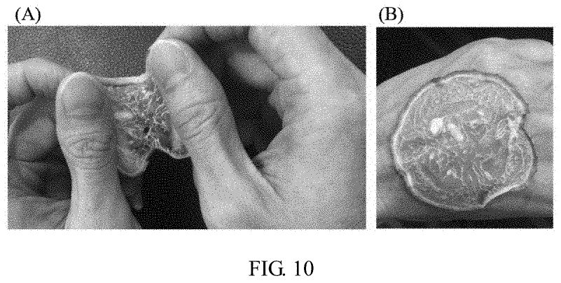

Three subjects are selected for skin testing. A freeze-drying loofah slice with a thickness of 2 mm is used as a mask substrate. The mask substrate is fully infiltrated with deionized water, and then applied to the skin of the subjects for 15 minutes. The result is shown in FIG. 10, which shows state photographs using a loofah slice as the mask substrate of the present disclosure. In FIG. 10, (A) is a state photograph when a subject takes out the infiltrated loofah slice; (B) is a state photograph when the slice is applied to the skin. When the subject is taken out the mask substrate, the loofah mask substrate is shown to be fully infiltrated, and it may not be easily torn when taken out and still maintain structural integrity. In addition, the loofah mask substrate may be easily applied to the skin without slipping randomly, and the fragrance of the loofah may be still maintained when applied. It is worth mentioning that the nutritional ingredients (e.g., tannins, flavonoids, alkaloids, vitamin E, vitamin C, palmitic acid, stearic acid, myristic acid, oleanolic acid, etc.) contained in the peel, pulp or seeds of the loofah may be easily released through the process of the present disclosure. Thus, the comfort of the skin and the increase in moisture retention may be felt after the application.

Efficacy of Using a Gherkin as a Mask Substrate

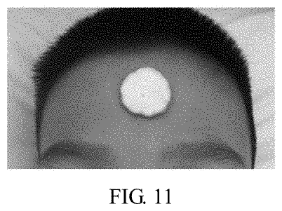

Three subjects are selected for skin testing. A freeze-drying gherkin slice with a thickness of 2 to 3 mm is used as a mask substrate. The mask substrate is fully infiltrated with deionized water, and then applied to the skin of the subjects for 15 minutes. The result is shown in FIG. 11, which shows a state photograph using a gherkin slice as the mask substrate of the present disclosure. The result shows that the gherkin mask substrate may be easily infiltrated and well applied to the skin without slipping randomly, and the fragrance of the gherkin may be still maintained when applied. It is worth mentioning that the comfort of the skin may be felt after the application. This may account for the fact that the nutritional ingredients (e.g., lactic acid, phosphatidylcholine, glutamic acid, phenols, vitamin C, arginine, aspartic acid, stearic acid, linoleic acid, oleic acid, palmitic acid, etc.) contained in the peel, pulp or seeds of the gherkin may be easily released through the process of the present disclosure. In addition, since the pores of the gherkin are denser, the texture thereof is more elastic than those of the loofah and cucumber.

Efficacy of Using a Cucumber as a Mask Substrate

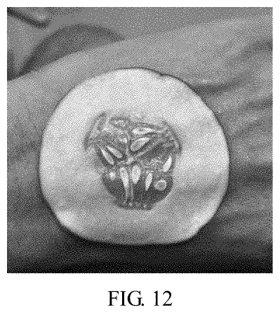

Two subjects are selected for skin testing. A freeze-drying cucumber slice with a thickness of 2 mm is used as a mask substrate. The mask substrate is fully infiltrated with deionized water, and then applied to the skin of the subjects for 15 minutes. The result is shown in FIG. 12, which shows a state photograph using a cucumber slice as the mask substrate of the present disclosure. The result shows that the cucumber mask substrate may be fully infiltrated, wherein the middle area may return to an original natural transparent film after infiltrated without being torn or becoming jelly and be well applied to the skin without slipping randomly. This has finer absorbency to the skin for a gherkin. When applied, the mask substrate still has the fragrance of a cucumber, and has a slight sense of smoothness. After applied for 15 minutes, a high-moisturizing state may still be maintained. The reason for a sense of smoothness felt by the skin may be the same as that of a cucumber. That is, the nutritional ingredients (e.g., lactic acid, phosphatidylcholine, glutamic acid, phenols, vitamin C, arginine, aspartic acid, stearic acid, linoleic acid, oleic acid, palmitic acid, etc.) contained in the peel, pulp or seeds of the cucumber may be easily released through the process of the present disclosure.

In summary, by the natural fiber structures, the mask substrate of the present disclosure may effectively absorb water or essence liquid. In the meantime, the active substances contained in the substrate may be released when applied, further improving the skin moisturizing and beauty effects. Furthermore, the mask substrate of the present disclosure is made from natural plant slices. Accordingly, the mask substrate may decompose naturally after use without causing environmental damage.

What is claimed is:

1. A mask substrate, comprising:
a substrate layer prepared by freeze-drying a plant slice having a predetermined thickness under a predetermined condition;
wherein after the mask substrate is rehydrated with an infiltrating liquid, a surface of the mask substrate is able to be applied to a skin of an individual.

2. The mask substrate according to claim 1, wherein the substrate layer has a thickness of 0.1 to 3 mm.

3. The mask substrate according to claim 1, wherein the predetermined condition for freeze-drying is a cavity temperature of 25° C. to −60° C., and a vacuum degree of 0 to 200 mT.

4. The mask substrate according to claim 1, wherein the plant slice is selected from a group consisting of white radish, taro, pumpkin, wax gourd, turnip, gherkin, cucumber, loofah, watermelon, pineapple, and carrot.

5. The mask substrate according to claim 1, wherein the substrate layer has pores in a size of 5 to 100 μm.

6. The mask substrate according to claim 1, wherein the mask substrate further comprises a packaging bag which seals the substrate layer therein.

7. The mask substrate according to claim 1, wherein the substrate layer is made without weaving, knitting, or non-woven fabric processing.

8. A mask kit, comprising a mask substrate in claim 1, and an infiltrating liquid, wherein the infiltrating liquid is deionized water or essence liquid;
wherein after the mask substrate is rehydrated with the infiltrating liquid, a surface of the mask substrate is able to be applied to the skin of an individual.
